SEQUENCE LISTING (6 sequences)

SEQ ID NO: 1 - DNA, 25 bp, Artificial Sequence
  Primer
gaagatctatgtcccctatactagg

SEQ ID NO: 2 - DNA, 24 bp, Artificial Sequence
  Primer
cgggatcccaggggcccctggaac

SEQ ID NO: 3 - DNA, 26 bp, Artificial Sequence
  Primer
cgggatcctctggaatatccctggac

SEQ ID NO: 4 - DNA, 26 bp, Artificial Sequence
  Primer
cggaattcgtgataaaaatagagttc

SEQ ID NO: 5 - DNA, 30 bp, Artificial Sequence
  Primer
cgggatccatgacttttaacagttttgaag

SEQ ID NO: 6 - DNA, 30 bp, Artificial Sequence
  Primer
cccaagcttttaagacataaaaattttttg

(19) United States
(12) Patent Application Publication
(10) Pub. No.: US 2009/0318376 A1
(43) Pub. Date: Dec. 24, 2009

(54) High Throughput Screening Method of Binding Inhibitor Between caspase3 and XIAP and Binding Inhibitor Screened Thereby

(75) Inventors: Bong Hyun Chung, Daejeon (KR); Kwang-Hee Son, Daejeon (KR); Moonil Kim, Daejeon (KR); Kyoungsook Park, Chungcheongnam-do (KR); Byoung-Mog Kwon, Daejeon (KR); Hyun Jung Junn, Daejeon (KR); Dong Cho Han, Daejeon (KR); Sun-Hee Jeon, Daejeon (KR)

Correspondence Address: MOORE & VAN ALLEN PLLC, P.O. BOX 13706, Research Triangle Park, NC, 27709, US

(73) Assignee: Korea Research Institute of Bioscience and Biotechnology, Daejeon (KR)

(21) Appl. No.: 12/304,766

(22) PCT Filed: Jun. 12, 2006
(86) PCT No.: PCT/KR07/02816

§ 371 Date: Jun. 24, 2009

(30) Foreign Application Priority Data
Jun. 15, 2006 (KR) 10-2006-0053922

(51) Int. Cl. A61K 31/702; G01N 33/573; C07H 15/24

(52) U.S. Cl. 514/33; 435/7.4; 536/18.1

(57) ABSTRACT

The present invention relates to a high throughput screening method of a binding inhibitor between caspase3 and xIAP and chromomycin screened using the same, and more specifically, the present invention provides a method for screening anticancer substance, the method comprising the steps of reacting caspase3 or xIAP and candidate inhibitors of the binding between caspase3 and xIAP on a biochip for detecting caspase3:xIAP interaction, and selecting a candidate substance inhibiting the binding of caspase3 to xIAP as an anticancer substance, and an anticancer agent inhibiting caspase3:xIAP binding, which is screened by the above method. According to present invention, it is possible to develop a target-oriented anticancer agent focused on xIAP and caspase3, apoptosis-related proteins and thus it can be applied to tailored medication and combination therapy. Moreover, glycoside antibiotic chromomycin, screened by the present invention has inhibitory activity of the binding between xIAP and caspase3 involved in apoptosis, so that it can be used as a therapeutic agent for myelogenous leukemia and solid tumors.

### TECHNICAL FIELD

The present invention relates to a high throughput method for screening an inhibitor of the binding between caspase3 and xIAP and an anticancer agent screened by the method.

### BACKGROUND ART

Selective anticancer agents, which act at a specific molecular target, draw much attention since they not only offer a safer and more efficient therapeutic method, but also can be applied to tailored medication and combination therapy. Apoptosis plays an important role in the elimination of unlimited cancer cell proliferation, which is the best target for cancer prevention theoretically. Although the process of apoptosis is very complicated, a core factor, such as caspase, Bcl, NF-kB has been identified. Particularly, caspase3, a main protein causing apoptosis, and Bcl-2 family and IAP (inhibitor of apoptosis protein) group, apoptosis inhibitor proteins, which are over-expressed in cancer cells, are emerging as a protein target which can be immediately applied in developing a new drug targeting cancer cells.

Apoptosis is a physiological process inherent in all cells, which is essential in development thereof and maintaining homeostasis within cell tissues. Apoptosis is an active process which is activated by an internal and external stimulation unlike necrosis. If strictly regulated apoptosis mechanism does not function or is not controlled properly, cancer, degenerative neurotic disease or other pathological condition will be developed. Therefore, apoptosis regulatory genes are useful gene group in developing not only anticancer medications but also drugs for protecting nerve cells.

Apoptosis is morphological and biochemical changes mediated by caspase, and two distinct pathways of caspase activation have been identified. Extrinsic pathway is initiated by binding a death ligand such as TNF-α, FasL to a death receptor (CD95, TNF receptor, TRAIL receptor) to activate initiating caspases neighboring cell membrane, and initiating caspases form cleavage to activate executing caspase such as caspase3,7. The other pathway activating caspase is an intrinsic pathway requiring the disruption of mitochondrial membrane and discharging of mitochondrial protein including Smac/DIABLO, HtRA2 and cytochrome c. Bax and Bid of Pro-apoptotic Bcl-2 family induce release of cytochrome c from mitochondrial inner membrane, and cytochrome c released into cytoplasm links to Apaf-1 (apoptotic protease activating factor-1) and ATP to activate caspase9 and thus initiate caspase cascade.

Studies on anticancer drugs related to apoptosis has been explaining the mechanism of a key protein, caspase based on phenomenological observation, such as DNA fragmentation, release of cytochrome c which is a hallmark of apoptosis.

In anticancer therapy, it has been known that administering a selective anticancer agent acting on a specific molecular target is safer and more effective since it can be applied in tailored medication and combination therapy. With the above known fact, it is in desperate need in the art to develop a method for effectively treating cancer by blocking a specific path in the cancer process.

Accordingly, the present inventors have made extensive efforts to develop a selective anticancer drug which acts on a specific molecular target existing during cancer progression, and as a result, developed a high throughput screening method of a key protein in apoptosis, caspase3, and an inhibitor of xIAP-binding inhibiting apoptosis, thereby completing the present invention.

### SUMMARY OF THE INVENTION

A main object of the present invention is to provide a method for screening an anticancer substance, which inhibits the interaction between caspase3 and xIAP.

Another object of the present invention is to provide an inhibitor of the binding between caspase3 and xIAP, screened by the method.

In order to accomplish the above object, the present invention provides a method for screening an anticancer substance, the method comprising the steps of:

(a) reacting (i) caspase3 or xIAP with (ii) candidate inhibitors of the binding between caspase3 and xIAP, on a biochip for detecting interaction between caspase 3 or xIAP, and

(b) selecting a candidate substance inhibiting the binding between capase3 and xIAP as an anticancer substance.

Moreover, the present invention provides an anticancer agent (glycoside antibiotics) screened by the above method, which has a structure of the following chemical formula 1 and inhibits the binding between capase3 and xIAP.

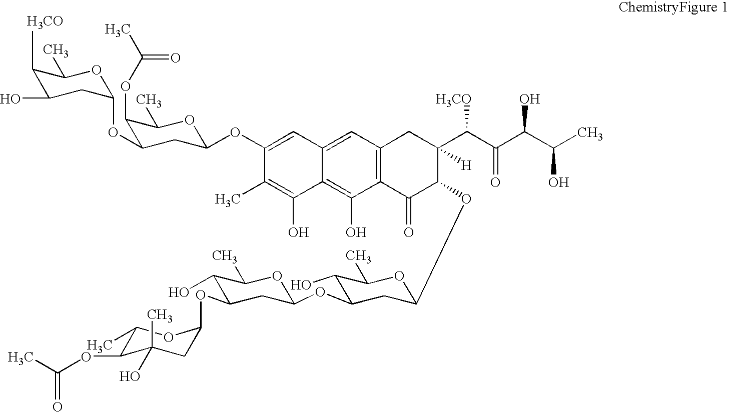

Another features and embodiments of the present invention will be more clarified from the following detailed description and the appended claims.

### BRIEF DESCRIPTION OF DRAWINGS

FIG. 1 is a photograph showing the result after examining whether caspase3 and xIAP are bound, using MBP (Maltose Binding Protein) resin and GST antibody.

FIG. 2 is a graphic diagram showing the inventive protein chip reacting with an active material concentration-dependently.

FIG. 3 is a graphic diagram showing the result of screening a material inhibiting interaction between caspase3 and xIAP.

FIG. 4 is a photograph showing the result of pull down assay, using tagged MBP to verify inhibition activity of the binding of caspase3 and xIAP by the screened chromomycin.

### DETAILED DESCRIPTION OF THE INVENTION, AND PREFERRED EMBODIMENTS

Apoptosis is a normal physiological process for embryo generation, tissue regeneration, and the like. Since cancer cells were generated by abnormal phenomenon of avoiding normal apoptosis, restoration of normal apoptosis will be the best mechanism for anticancer. Caspase3 plays a key role in apoptosis, and the activation of caspase3 can be inhibited by inhibitors of apoptosis proteins such as xIAP. With reports on over-expression of xIAP in a significant number of anticancer cells, xIAP has been suggested as an anticancer target (Aaron D. Sxhimmer et al., Cell, 5:25, 2004)

Accordingly, the present inventors have made extensive efforts to develop a method for screening an inhibitor of the binding between caspase3 and xIAP, and as a result, have completed the present invention.

In one aspect, the present invention relates to a method for screening an anticancer substance, the method comprising the steps: (a) allowing (i) caspase3 or xIAP and (ii) a candidate inhibiting the binding between caspase3 and xIAP, on a biochip for detecting interaction between caspase3 or xIAP, and (b) selecting a candidate substance inhibiting the binding between capase3 and xIAP, as an anticancer substance.

In the present invention, the biochip for detecting interaction between the caspase3 (caspase3) and xIAP is preferably a biochip having caspase3 or xIAP immobilized on a substrate thereof.

In the present invention, the candidate inhibitors of the binding between the caspase3 and xIAP are preferably analyzed by SPRI (Surface Plasmon Resonance Imaging), fluorescent substances, or radioactive isotopes, but it is not limited thereto.

The inhibitor of the binding between caspase3 and xIAP, screened according to the above method, is a Chromomycin (C57H82026, M.W. 1183.26) which is a glycoside antibiotic and has a structure of chemical formula 1. Chromomycin is a glycoside antibiotic, which is known to have a mechanism of nucleic acid binding (Shuhei Imoto et al., Bioorg. Med. Chem. Lett., 14:4855, 2004) by attaching to the small groove of deoxyribonucleic acid, and thus be used as a probe detecting nucleic acid.

According to the present invention, Chromomycin first binds to xIAP to block protein-protein interactions resulting in the binding between xIAP and caspase3, thereby acting as an anticancer agent, suggesting that it can be applied for target-oriented anticancer agents regulating an apoptosis-related mechanism. Also, chromomycin, glycoside antibiotics was not used frequently in the prior art due to its high toxicity. However, since the new mechanism of chromomycin is found in the present invention, increasing use of chromomycin is expected.

As a result of measuring inhibitory activity of Chromomycin(chemical formula 1) in cancer cell lines, IC50 value was 8.4 nM for breast cancer cell line MDA-MB-231 (ATCC HTB-26, USA), and 6.7 nM for colorectal cancer cell line HCT116 (ATTC CCL-247, USA), which is a strong inhibitory activity; and 80 nM for colorectal cancer cell line SW620 (ATCC CCL-227, USA). Accordingly, the Chromomycin represented by chemical formula 1, which inhibits protein-protein interaction between caspase3 and xIAP, can be used as an anticancer therapeutic agent inducing apoptosis of cancer cells.

### Examples

Hereinafter, the present invention will be described in more detail by examples. It will be obvious to a person skilled in the art, however, that these examples are for illustrative purpose only and are not construed to limit the scope of the present invention.

Especially, in the following examples, among candidate inhibitors of the binding between caspase3 and xIAP, selected by analyzing with SPRL (Surface Plasmon Resonance Imaging) although the inhibition of the binding between caspase3 and xIAP and the inhibition of cancer cell proliferation by glycoside antibiotic, Chromomycin were explained, it is obvious to a person skilled in the art that inhibitors screened by the inventive method for screening an inhibitor of the binding between caspase3 and xIAP have the same effect as glycoside antibiotics, through the detailed description.

### Example 1

### Construction of a Protein Chip for Screening an Inhibitor of the Binding between Caspase3 and xIAP

1-1: Construction of Recombinant Clone of Caspase3 Gene and Protein Expression

cDNA library, which was prepared from total RNA extracted from a human cell line using a reverse transcriptase, was used as a template, and primers of SEQ ID NOs: 1 and 2 were designed such that they contain restriction enzymes BglII and BamHI for cloning, and primers of SEQ ID NOs: 3 and 4 were designed such that they contain BamHI and EcoRI for cloning. Herein, the designed primers are specific to a sequence of the human caspase3 gene.

SEQ ID NO: 1

(sense): 5′GAAGATCTATGTCCCCTATACTAGG-3

SEQ ID NO: 2

(antisense): 5′CGGGATCCCAGGGGCCCCTGGAAC-3

SEQ ID NO: 3

(sense): 5′CGGGATCCTCTGGAATATCCCTGGAC-3

SEQ ID NO: 4

(antisense): 5′CGGAATTCGTGATAAAAATAGAGTTC-3

Glutathione-S transferase (GST) gene was amplified by PCR using the primers of SEQ ID NOs: 1 and 2 to obtain 735 bp GST and the GST gene was amplified using the primers of SEQ ID NOs: 3 and 4 to obtain 747 bp Cas3 (29), and the amplified genes were treated with the corresponding restriction enzyme, respectively, then inserted into pET-GST vector (Novagen, USA) digested with the same restriction enzymes thus constructing pET-GST-Cas3 (29) vector.

E. coli BL 21 (Novagen, USA) was transformed with the prepared pET-GST-Cas 3 (29) vector and shaking-cultured in 2× TY culture medium(Tryptone 1.6%, yeast extract 1%, Nacl 0.5%) at 37° C. to an optical density of 0.6 (A600 nm); followed by adding IPTG (isopropyl β-D-thiogalactopyranoside) to a final concentration of 0.4 mM, to induce protein expression at 37° C., thus obtaining protein solution by ultrasonic wave disruption and centrifugation.

The protein solution mixed with a buffer solution (12 mM Tris-Cl, pH 6.8, 5% glycerol, 2.88 mM mercaptoethanol, 0.4% SDS, 0.02% bromophenol blue) was heated at 100° C. for 4 minutes and loaded onto a polyacrylamide gel having 5% gel (pH 6.8, width 10 cm, height 12.0 cm) covered on 10% a separation gel with thickness of 1 mm (pH 8.8, width 20 cm, height 10 cm), followed by electrophoresis at 200-100V, 25 mA for 1 hour to stain with a Solution of Coomassie Blue, thus confirming recombinant protein.

1-2: Expression and Purification of xIAP

In order to tag MBP (Maltose Binding Protein) to the N-terminus of human xIAP gene, PCR was carried out using cDNA library, prepared from total RNA extracted from a human cell line using a reverse transcriptase, as a template and primers of SEQ ID NOs: 5 and 6 specific to a base sequence of human xIAP gene, which is designed such that they contain BamHI and HindIII to obtain a PCR product (1,494 bp). Then, the obtained PCR product was treated with restriction enzymes, respectively and inserted into vector pMAL-c2x (New England Biolabs, UK) digested with the same restriction enzymes, thus constructing pMAL-xIAP for recombinant protein.

SEQ ID NO 5

(sense): 5′CGGGATCCATGACTTTTAACAGTTTTGAAG-3

SEQ ID NO 6

(antisense): 5′CCCAAGCTTTTAAGACATAAAAATTTTTTG-3

E. coli BL21 transformed with the prepared pMAL-xIAP was shaking-cultured in an LB medium to obtain a whole protein solution using the same method as described in Example 1-1, thus confirming recombinant protein by electrophoresis and staining with a Solution of Coomassie Blue.

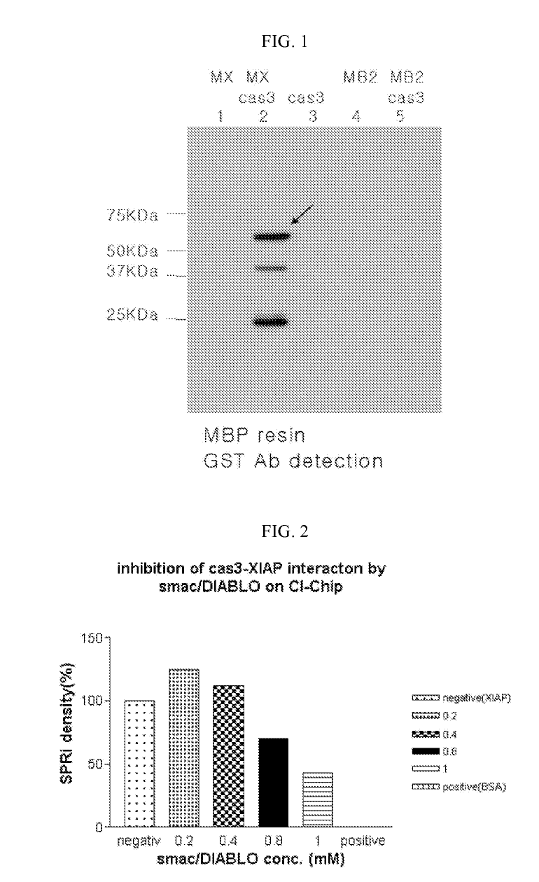

Caspase3 and xIAP, expressed and obtained in Examples 1-1 and 1-2, were confirmed as normal proteins binding to each other by using MBP resin and GST antibody (FIG. 1). As a result, as shown in FIG. 1, a binding protein of caspase3 and xIAP was detected around 60 KDa.

1-3: Construction of Caspase3-xIAP Chip (CI-Chip)

1-3-1: Construction of a Gold Chip Coated with Gold Thin Film

To construct a gold chip coated with gold thin film, a binding agent, chromium (Cr) was coated to have the thickness of 2 nm on a thin glass substrate (22 mm×22 mm×0.3 t) using commercially available electron-beam evaporator (Dada Inc, Korea) to attach 47-nm-thick gold thin film, thus constructing a gold chip. The gold chip, obtained by coating gold thin film on the glass substrate, was treated with Piranha solution (70% H₂SO₂, 30% H₂O₂) at 65° C. for 30 minutes and immersed in 10 nM of 11-mercapto-1-undecanoic acid (MUA) solution dissolved in ethanol for 16 hours, thus forming self assembled monolayers (SAMs).

Also, in order to activate the gold chip having the formed self assembled monolayers, 0.4M sodium hydroxide solution was mixed with 2-methoxy-ethyl-ether solution in a volume ratio of 1:1 and the mixture solution was added with ephichlorohydrin to a concentration of 0.6M, and then allowed to react with the gold chip at the room temperature for 4 hours. The activated chip surface was coated with dextran by allowing the activated chip surface to react with 0.3mg/ml of dextran solution dissolved in 0.1M sodium hydroxide solution at the room temperature for 20 hours. Dextran surface was activated with ephichlorohydrin using the same method as described above, immersed in 44 mM reduced L-glutathione (GSH) dissolved in 100 mM phosphate buffer solution (pH7.0) and allowed to react at 37° C. for 20 hours, followed by discarding the reaction solution to wash the chip with distilled water. Also, in order to remove a non-reacting active group on the chip surface, 1M ethanolamine solution was treated on the chip surface and allowed to react at 37° C. for 4 hours.

1-3-2: Immobilization of GST-Tag Protein on the Gold Chip Surface

Whether the obtained proteins bind to the gold chip having L-glutathione on the surface thereof, was pre-experimented. After a bacteria cell extract containing 20% glycerol, in which GST-tag-fused cas3 (29) obtained in Example 1-1 has been over-expressed, was dispersed in 384 microwell plate, it was spotted on the gold chip having L-glutathione on the surface thereof with a pin having a diameter of 335 μm under a constant humidity of 75% using automatic robotic arrayer(proteogen, CM-1000), and then subjected to RT shaking incubation for 3 hours, thus constructing Cl-chip having gold chip as a substrate. In order to examine whether the Cl-chip, constructed by the above method, work, BSA as a negative control and Smac/DIABLO, xIAP-inhibiting peptide as a positive control were subjected to screening, thus confirming that the constructed protein chip reacts to an active substance concentration-dependently (FIG. 2). As a result, as shown in FIG. 2, it was found that the constructed protein chip (CI-chip) reacts with an active substance concentration-dependently.

### Example 2

### Screening of Inhibitor of the Binding between Caspase3 and xIAP

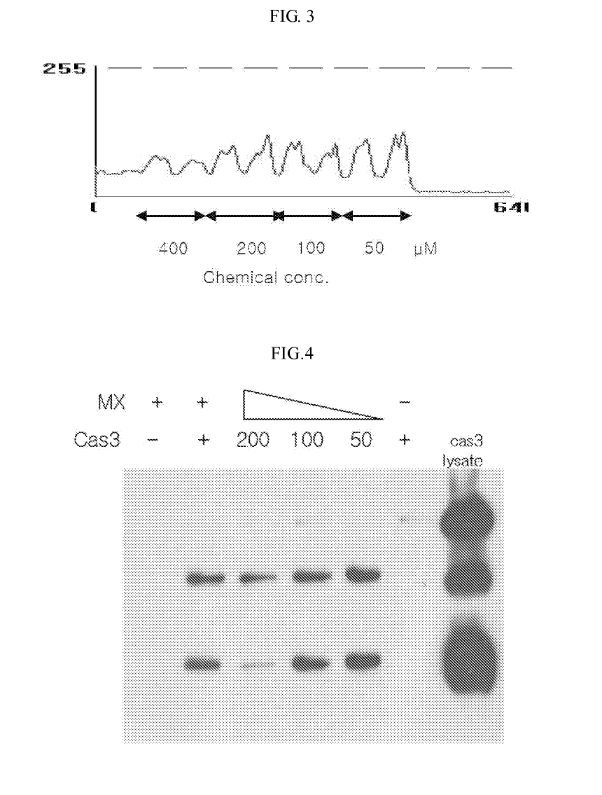

Inhibitors of interaction between caspase3 and xIAP were screened using the protein chip based on the gold chip prepared in example 1. 920 microorganism-derived samples, which was purely isolated or partially purified, were reacted with 15 μl solution (3.75 μl of more than 50% glycerol; 3 μl of material dissolved in DMSO at a concentration of less than 10 mg/ml; 7.5 μl of xIAP dissolved in PBS at a concentration of more than 1 mg/ml; 0.75 μl of PBS buffer) at 4° C. for 2 hours to apply to the protein chip. Inhibitory activity was analyzed using SPR imaging as the concentration of protein in solution was increased (FIG. 3).

As a result, chromomycin was found as hit molecule, and as shown in FIG. 3, it showed constant inhibition reaction concentration-dependently in the protein chip within the range from 50 to 400 μm, thereby confirming that it is an inhibitor of interaction between caspase3 and xIAP.

### Example 3

### Inhibition of the Binding between Caspase3 and xIAP by Chromomycin

In order to examine whether chromomycin, screened in the example 2, is activated by the reaction of caspase3 and xIAP, pull down assay using tagged MBP (Maltose Binding Protein) was performed. After xIAP and chromomyicin were added to MBP resin and allowed to react on ice for 2 hours, the resulting mixture was added with caspase3 and allowed to react for about 3 hours, and then washed with PBS buffer solution to load onto 10% SDS PAGE, thus examining the reactant (FIG. 4).

As a result, as shown in FIG. 4, it was found that the resulting reactant of xIAP (MX) and caspase3 (Cas3) was reduced, as chromomycin was increased from 50 μm to 200 μm.

### Example 4

### Inhibition of Proliferation of Cancer Cell Lines by Chromomycin

To examine inhibition of proliferation of the cell lines, the cancer cell lines HCT 116 (Mecoy's 5A medium), SW620 (RPMI 1640) and MDA-MB-231 (RPMI 1640) were cultured to disperse in a 96 well plate. SW620 and HCT116 were innoculated at a concentration of 7,000 cells/100 μl, and MDA-MB-231 was innoculated at a concentration of 5,000 cells/100 μl, respectively, and cultured overnight, followed by an additional reaction of over 24 hours after treating with 1 μl chromomycin prepared according to the concentration. Next, the resulting cells were added with 10 μl of WST-8, counted with an ELISA reader at 450 nm after 2 hours and calculated as a percentage of comparative control added only with DMSO. Inhibitory concentration (IC50), with respect to proliferation of cell lines MDA-MB -231, HCT116, SW620, was measured within the concentration range from 0 to 1 82 g/ml by dissolving chromomycin in DMSO (Table 1).

As a result, as shown in table 1, breast cancer cell line MDA-MB-231 (ATCC HTB-26, USA) has 8.4 nM of IC 50 value, colorectal cancer cell line HCT 116 (ATCC CCL-247, USA) has 6.7 nM, which is strong inhibitory activity, and colorectal cancer cell line SW 620 (ATCC CCL-227, USA cell line) has 80 nM.

TABLE 1

| Cell host | IC 50(nM) |
|---|---|
| MDA-MB-231 | 8.4 |
| HCT116 | 6.7 |
| SW 620 | 80 |

### Industrial Applicability

As described in detail above, the present invention has an effect to provide a high throughput screening method of a binding inhibitor between Caspase3 and xIAP and chromomycin screened using the same. According to the present invention, it is possible to develop target-oriented anticancer agent focused on xIAP and caspase3, apoptosis-related proteins and thus it can be applied to tailored medication and combination therapy. Moreover, glycoside antibiotic chromomycin, screened by the present invention has inhibitory activity of the binding between xIAP and caspase3 involved in apoptosis, so that it can be used as a therapeutic agent for myelogenous leukemia and solid tumors.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### Sequence Listing

Electric Attachment

1. A method for screening an anticancer substance, the method comprising the steps of:
(a) reacting (i) caspase3 or xIAP with (ii) candidate inhibitors of the binding between caspase3 and xIAP, on a biochip for detecting interaction between caspase3 or xIAP, and (b) selecting a candidate substance inhibiting the binding between capsase3 and xIAP as an anticancer agent.

2. The method for screening an anticancer substance according to claim 1, wherein the biochip for detecting interaction between caspase 3 or xIAP is a biochip having caspase3 or xIAP fixed on a substrate thereof.

3. The method for screening an anticancer substance according to claim 1, wherein the candidate inhibitors of the binding between caspase3 and xIAP are analyzed by SPRI (Surface Plasmon Resonance Imaging).

4. The method for screening an anticancer substance according to claim 1, wherein the inhibitors of the binding between caspase3 and xIAP are analyzed using fluorescent substances, or radioactive isotopes.

5. An anticancer agent inhibiting the binding between caspase 3 and xIAP, which is represented by the following chemical formula 1.

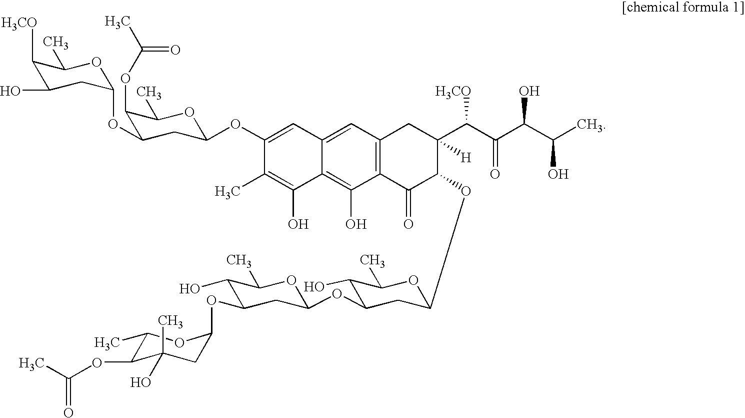

6. A method for treating cancer using the anticancer agent according to claim 5.
